(19) United States
(12) Patent Application Publication
(10) Pub. No.: US 2025/0065031 A1
(43) Pub. Date: Feb. 27, 2025

(54) CARRY CASE FOR A DRUG DELIVERY DEVICE AND DRUG DELIVERY DEVICE

(71) Applicant: Sanofi, Paris (FR)

(72) Inventors: Matthias Felber, Wil (CH); Florian Eberli, Wil (CH); Adrian Geiger, Wil (CH)

(21) Appl. No.: 18/727,607

(22) PCT Filed: Jan. 9, 2023
(86) PCT No.: PCT/EP2023/050257

§ 371 Date: Jul. 9, 2024

(30) Foreign Application Priority Data
Jan. 10, 2022 (EP) 22315006.1

(51) Int. Cl. A61M 5/00; A61M 5/315

(52) U.S. Cl. CPC A61M 5/002; A61M 5/31535; A61M 5/31553; A61M 2205/103; A61M 2205/3561; A61M 2205/3584; A61M 2205/586; A61M 2209/06

(57) ABSTRACT

A carry case for storing a drug delivery device and to a drug delivery device having a rotatable dose dial member. The case includes an outer shell, and a support recess for receiving and retaining the drug delivery device. In addition, the case further includes a coupling member for mechanically engaging the dose dial member of the drug delivery device, an electrically driven motor, a battery, and a processor connected with the motor and the battery. The coupling member is configured to be driven by the motor to rotate the dose dial member when engaged with the coupling member.

### CROSS-REFERENCE TO RELATED APPLICATIONS

This is a National Stage Application, filed under 35 U.S.C. § 371, of International Patent Application No. PCT/EP2023/050257, filed on Jan. 9, 2023, which claims priority to European Patent Application No. 22315006.1, filed on Jan. 10, 2022, the disclosures of which are incorporated herein by reference in their entireties.

### TECHNICAL FIELD

The present invention is generally directed to a carry case for a drug delivery device. The present invention further relates to a drug delivery device and to a system of such a carry case and a corresponding drug delivery device.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament. However, setting the correct dose amount may be difficult or burdensome for e.g., visually and/or manually impaired patients.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present invention is applicable for disposable and reusable devices.

It is generally known to store drug delivery devices in a carry case which prevents damaging the device or preventing contamination of the device. Such a carry case may comprise an outer shell and a support recess for receiving and retaining the drug delivery device. DE 42 08 677 A1 discloses an injection device for visually impaired patients comprising a case with a receiving section for a pen-type drug delivery device, a dose setting wheel, and a gear wheel coupled to the dose setting wheel. The dose setting wheel is configured to engage with an annular gear wheel on the drug delivery device when the drug delivery device is retained in the case.

### SUMMARY

It is an object of the present disclosure to facilitate dose setting especially for visually and/or manually impaired patients.

This object is solved for example by the subject matter defined in the independent claims. Advantageous embodiments and refinements are subject to the dependent claims. However, it should be noted that the disclosure is not restricted to the subject matter defined in the appended claims. Rather, the disclosure may comprise improvements in addition or as an alternative to the ones defined in the independent claims as will become apparent from the following description.

One aspect of the disclosure relates to a carry case with an outer shell and a support recess for receiving and retaining the drug delivery device. In addition, the case comprises a coupling member for mechanically engaging a rotatable dose dial member of the drug delivery device. The coupling member may comprise and/or may be part of a gearbox mechanism. Further, the case may comprise an electrically driven motor, an electrical power supply, e.g., one or more batteries, like a coin cell type battery or a cylindrical battery, and a processor connected with the motor and the battery. The processor may be provided with or may comprise a memory for storing data.

Preferably, the coupling member is configured to be driven by said motor to rotate the dose dial member when engaged with the coupling member. In other words, the case is configured and adapted to perform a dose setting operation by actuating the dose dial member of the drug delivery device. This dose setting operation may be the only possibility of dose setting for the drug delivery device or may be an additional function as an alternative to the known manual dose setting operation. Thus, in addition to storing and protecting the drug delivery device, the case has an additional function allowing to set a dose to be administered by the drug delivery device.

The case may be generally suitable to store and retain any kind of, e.g., pen-type, drug delivery device and its accessory, like injection needles. On the other hand, the case according to the present disclosure is adapted to mate and interact with a certain type of drug delivery devices, namely drug delivery devices comprising a rotatable dose dial member for setting a dose to be administered by the drug delivery device wherein the dose dial member is mating with the coupling member of the case to transfer torque from the coupling member to the dose dial member.

According to an example, the coupling member may be a gear wheel, especially a gear wheel with a series or gear teeth facing radially outwards. The motor may be a micro gear-motor, i.e., a combination of a motor and a gear box, wherein the gear box may comprise the coupling member or may be engageable by the coupling member.

The case may further comprise a communication unit for communicating with another device, e.g., a wireless communication interface for communicating with another device via a wireless network such as Wi-Fi or Bluetooth, or even an interface for a wired communications link, such as a socket for receiving a Universal Series Bus (USB), mini-USB or micro-USB connector. Preferably, the case comprises an RF, WiFi and/or Bluetooth unit as the communication unit. The communication unit may be provided as a communication interface between the case and the exterior, such as other electronic devices, e.g., mobile phones, personal computers, laptops and so on. For example, dose data may be transmitted by the communication unit to the external device. The dose data may be used for a dose log or dose history established in the external device. In addition or as an alternative, the communication unit may receive data from the external device, e.g., data regarding a health condition of a user and/or dose data regarding an amount of drug to be delivered by the drug delivery device.

In addition or as an alternative to the communication unit, the case may comprise at least one data input member and/or at least one data output member. For example, dose data may be entered by means of at least one data input member. A data output member may comprise an LCD-display connected to the processor, e.g., to show the history of last administered doses or a dose recommendation.

In order to facilitate setting of a dose in a drug delivery device using the case of the present disclosure, the processor may be configured to calculate, receive and/or store data corresponding to a dose amount to be administered by the drug delivery device. In more detail, the processor may be configured to energize the motor for rotating the coupling member a defined number of revolutions corresponding to a dose amount to be administered by the drug delivery device. This may include a rotation of the coupling member in a dose decreasing direction until any previously set dose is cancelled, followed by a rotation of the coupling member in a dose increasing direction until the intended dose is set. In order to more accurately perform a defined number of rotations with a motor, for example a miniature DC motor with gearbox, the motor or mechanism may comprise a position sensor and/or encoder allowing to operate the motor in a closed-loop positioning mode. Such a position sensor and/or encoder may be coupled or connected to the processor.

According to a still further aspect of the present disclosure, a drug delivery device for setting and dispensing, e.g., variable or fixed, doses of a liquid drug may comprise a cartridge containing a liquid drug and a dose setting and drive mechanism which is configured to perform a dose dialing operation for selecting a dose to be delivered by the drug delivery device and a dose delivery operation for delivering the set dose wherein the dose setting and drive mechanism comprises a rotatable dose dial member, e.g., configured to be mechanically engaged by a driven coupling member preferably by the coupling member of the above described case. In an example, the dose dial member comprises a ring of radially inwardly directed gear teeth.

The drug delivery device may be a reusable device permitting replacement of an empty cartridge. For example, the cartridge may be received in a releasably attached cartridge holder.

In one embodiment, the drug delivery device comprises a dial sleeve, e.g., a number sleeve, which is rotatable relative to a housing, e.g., along a helical path, at least in the dose setting operation. In addition, a manually operable injection trigger, for example a dose and/or injection button or a member axially and/or rotationally locked thereto may be axially displaceable relative to the dial sleeve and rotationally constrained to the housing at least in the dose delivery operation.

The present disclosure is applicable for devices which are manually driven, e.g., by a user applying a force to an injection button, for devices which are driven by a spring or the like and for devices which combine these two concepts, i.e., spring assisted devices which still require a user to exert an injection force. The spring-type devices involve springs which are preloaded and springs which are loaded by the user during dose selecting. Some stored-energy devices use a combination of spring preload and additional energy provided by the user, for example during dose setting.

According to an aspect of the present disclosure, the dose dial member may be located in a proximal end portion of the drug delivery device. In order to prevent unintended amendment of the set dose, at least the radial outer surface and/or a proximally facing annular surface of the dose dial member may be shielded by a cover member of the drug delivery device.

In addition or as an alternative, the drug delivery device may further comprise a locking mechanism which may be switched between a locked state preventing dose setting and a released state permitting dose setting. For example, the locking mechanism prevents rotation of the dose dial member when in its locked state and permits rotation of the dose dial member when in its released state. Optionally, the locking mechanism may be biased into its locked state and may be switched into its released state by placing the drug delivery device in the case, especially by mechanical engagement of the dose dial member with the coupling member. In an alternative example, an axially shiftable part, matching the inner geometry of the dial member, may lock the rotation, and can then be pushed in by the coupling member, thereby releasing the rotation. As a still further alternative, the inwardly facing gears could be used as an axially movable locking member and a gear-wheel with outwardly facing teeth may be used as the dial member. In this case, the coupling member may have a matching annular shape with inwardly facing teeth. Still further, the drug delivery device may comprise an annular slot, for example below the cover, and a matching geometry of the case may enter this slot as the device is put into the case to thereby unlock the mechanism.

The present disclosure is further directed to a combination or system comprising a carry case and a drug delivery device as described above.

The present disclosure further pertains to a drug delivery device with the electronic system as described above which drug delivery device comprises a cartridge containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber.

In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human B30-N-myristoyl-ThrB29LysB30 insulin; human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR 709, ZP-2929, ZP-3022,

ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g., the axis which extends through the proximal and distal ends of the cartridge, the cartridge holder or the drug delivery device.

### BRIEF DESCRIPTION OF THE DRAWINGS

Non-limiting, exemplary embodiments of the disclosure will now be described with reference to the accompanying drawings, in which:

FIG. 1 is a perspective view of an embodiment of a carry case and a corresponding drug delivery device; and

FIG. 2 shows a detail of the case and the drug delivery device.

In the figures, identical elements, identically acting elements or elements of the same kind may be provided with the same reference numerals.

### DETAILED DESCRIPTION

In the following, some embodiments will be described with reference to an insulin injection device. The present disclosure is however not limited to such application and may equally well be deployed with injection devices that are configured to eject other medicaments or drug delivery devices in general, preferably pen-type devices and/or injection devices.

Certain embodiments in this document are illustrated suitable to be used with an injection device as described in WO 2014/033195 or WO 2014/033197 where an injection button and grip (dose setting member or dose setter) are combined. The injection button may provide the user interface member for initiating and/or performing a dose delivery operation of the drug delivery device. The grip or knob may provide the user interface member for initiating and/or performing a dose setting operation. Both devices are of the dial extension type, i.e., their length increases during dose setting. Other injection devices with the same kinematical behavior of the dial extension and button during dose setting and dose expelling operational mode are known as, for example, the Kwikpen® device marketed by Eli Lilly (also described in WO 2005/018721) and the Novopen® 4 device marketed by Novo Nordisk (also described in U.S. Pat. No. 6,663,602). An application of the general principles to these devices therefore appears straightforward and further explanations will be omitted. However, the general principles of the present disclosure are not limited to that kinematical behavior. Certain other embodiments may be conceived for application to an injection device as described in WO 2004/078239 or WO 2009/132777 where there are separate injection button and grip components/dose setting members. Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

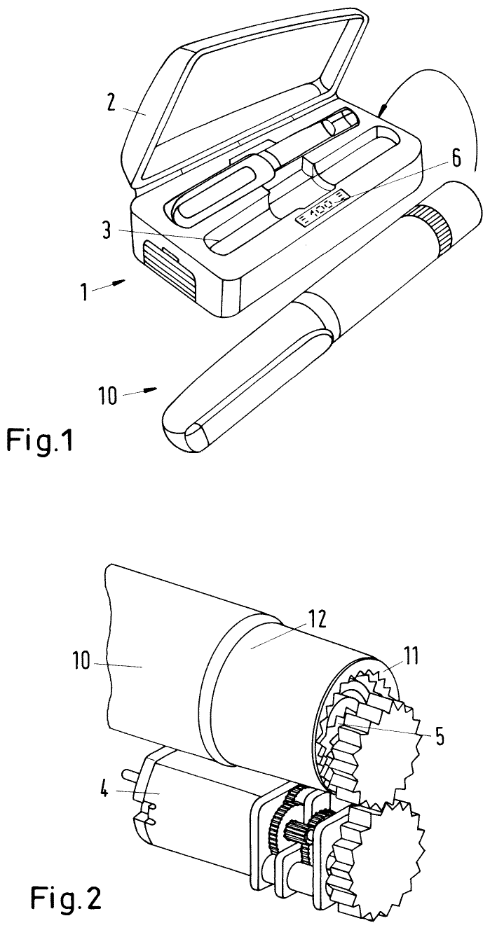

FIG. 1 is a perspective view of a carry case 1 for storing at least one drug delivery device 10. The case 1 comprises an outer shell 2 consisting of two hinged half-shells in the depicted exemplary embodiment. The half-shells may be opened as depicted in FIG. 1 to allow access to the interior of the shell 2 or may be closed to encase the at least one drug delivery device 10.

At least one support recess 3 is provided in the shell 2 formed to receive a pen-type drug delivery device 10. For this purpose, the support recess has a shape for snuggingly receiving at least a portion of the drug delivery device 10. For example, the drug delivery device 10 may be retained by snap engagement in the support recess 3.

Further, the case 1 comprises an electrically driven motor 4, a coupling member in the form of a gear wheel 5 connected to the motor 4, a data input means, e.g., an LCD-display 6, a battery (not shown) and a processor (not shown) connected with the motor 4, the display 6 and the battery. The case may optionally comprise data input means (not shown), e.g., a Bluetooth® communication interface and/or a keyboard, and/or a socket for connecting the processor or battery of the case 1 with external devices.

As shown in FIG. 2 in more detail, the coupling member is a gear wheel 5 with a series of gear teeth facing radially outwards. The gear wheel 5 may be part of a gear box rotationally connected to the motor 4 to drive the gear wheel 5 as the motor 4 is operated. The gear wheel 5 mates and engages with a rotatable dose dial member 11 of the drug delivery device 10 when the drug delivery device 10 is received in the support recess 3 of the case 1.

The dose dial member 11 is located at the proximal end of the drug delivery device 10 and comprises gear teeth facing radially inwards in the depicted example. As alternatives, the dose dial member 11 may comprise gear teeth facing outwards, or a geometry on the distal surface which allows coupling in axial direction, e.g., teeth or holes, with corresponding pins as coupling members. A cover 12 is provided as a part of the drug delivery device 10 at least partially shielding dose dial member 11. In the depicted example, cover 12 shields a radial outer surface of the dose dial member 11 and an annular proximal surface of the dose dial member 11, thereby preventing access to the dose dial member 11 from the lateral and proximal sides. If, as shown in the example of FIG. 2, the distal face of the dose dial member 11 is shielded by the adjacent component(s) of the drug delivery device 10, the gear teeth of the dose dial member 11 are only accessible from a radially inner side, e.g., by gear wheel 5 as depicted. This arrangement prevents unintended or undesired manipulation of the dose dial member 11.

In addition or as an alternative, the drug delivery device 10 may be provided with a locking mechanism (not shown) which may be switched between a locked state preventing dose setting and a released state permitting dose setting. In more detail, such a locking mechanism may prevent undesired rotation of the dose dial member when in its locked state and permits rotation of the dose dial member, especially during dose setting, when in its released state.

In an exemplary embodiment, the locking mechanism may be biased into its locked state. This means that in an idle condition of the drug delivery device 10, i.e., without a user applying any force or torque to the device, the locking mechanism is in its locked state. This may be due to a spring holding the locking mechanism in its locked state or a locking member may be snapped into the locked state. On the other hand, the locking mechanism may be switched into its released state either automatically, e.g., by placing the drug delivery device in the carry case 1, and/or by actuation of a user. In an example, the locking mechanism may be switched into its released state by mechanical engagement of the dose dial member 11 with the coupling member, i.e., the gear wheel 5. As an alternative, a clamping feature retaining the drug delivery device 10 in the case 1 may switch the locking mechanism into its released state when the drug delivery device 10 is received in the support recess 3 of carry case 1.

The battery powered case 1 may be configured to automatically, i.e., without manual operation of a user, and/or autonomously, i.e., without requiring using a smart phone or the like external device, set a dose in the drug delivery device 10. This results in high dose accuracy during dose setting and facilitates use for manually and/or visionally impaired patients. In addition, the drug delivery device 10 may be a fully mechanical device and may be made simple, e.g., even without a dose display.

For example, the case 1 may be configured to set a dose based on dose information stored and/or calculated in the case 1. The dose is set by operating motor 4 such that the gear wheel 5 is rotated for a predetermined number of rotations corresponding to the intended dose to be set. In order to more accurately perform a defined number of rotations with the type of motor shown in FIG. 2, i.e., a miniature DC motor with gearbox, the motor or mechanism may comprise a position sensor, i.e., an encoder (not shown), allowing to operate the motor in a closed-loop positioning mode. In such a scenario, it may be desirable that the gear wheel 5 is not accessible by the patient and/or that the set dose is locked after removing the drug delivery device 10 from the case 1. As an example, the drug delivery device 10 may be designed not having any accessible moving parts except for an injection trigger (not shown).

The processor of the case 1 may further be configured to calculate, receive and/or store data corresponding to a dose amount to be administered by the drug delivery device 10. This may include the function of a dose coach selecting the required dose based on health data or other patient data received and/or stored in the case 1 and/or transmitted to the case 1 by an external device.

### Reference Numerals

1 carry case
2 outer shell
3 support recess
4 motor
5 coupling member (gear wheel)
6 display
10 drug delivery device
11 dose dial member
12 cover

1. A carry case for storing a drug delivery device having a rotatable dose dial member, the carry case comprising:
an outer shell;
a support recess for receiving and retaining the drug delivery device;
a coupling member for mechanically engaging the rotatable dose dial member of the drug delivery device;
an electrically driven motor;
a battery; and
a processor connected with the electrically driven motor and the battery;
wherein the coupling member is configured to be driven by said electrically driven motor to rotate the rotatable dose dial member when engaged with the coupling member.

2. The carry case according to claim 1, wherein the coupling member is a gear wheel.

3. The carry case according to claim 1, wherein the electrically driven motor is a micro gear-motor.

4. The carry case according claim 1, further comprising a communication unit connected to the processor for communicating with another device.

5. The carry case according to claim 4, wherein the communication unit comprises a wireless communication interface for communicating with the another device via a wireless network.

6. The carry case according to claim 1, further comprising an LCD-display connected to the processor.

7. The carry case according to claim 1, wherein the processor is configured to calculate, receive and/or store data corresponding to a dose amount to be administered by the drug delivery device.

8. The carry case according to claim 7, wherein the processor is configured to energize the electrically driven motor in a closed-loop positioning mode based on signals from a positioning sensor and/or encoder, for rotating the coupling member a defined number of revolutions corresponding to a dose amount to be administered by the drug delivery device.

9. A drug delivery device for setting and dispensing liquid drug, the drug delivery device comprising a cartridge containing the liquid drug and a dose setting and drive mechanism which is configured to perform a dose dialing operation for selecting a dose to be delivered by the drug delivery device and a dose delivery operation for delivering a set dose, the dose setting and drive mechanism comprising a rotatable dose dial member configured to be mechanically engaged by a driven coupling member of a carry case.

10. The drug delivery device according to claim 9, wherein the rotatable dose dial member comprises a ring of radially inwardly directed gear teeth.

11. The drug delivery device according to claim 9, wherein the rotatable dose dial member is located in a proximal end portion of the drug delivery device and wherein at least a radial outer surface and/or a facing annular surface of the rotatable dose dial member is shielded by a cover member of the drug delivery device.

12. The drug delivery device according to claim 9, wherein the dose setting and drive mechanism comprises a manually operable injection trigger.

13. The drug delivery device according to claim 9, further comprising a locking mechanism which may be switched between a locked state preventing dose setting and a released state permitting dose setting.

14. The drug delivery device according to claim 13, wherein the locking mechanism prevents rotation of the rotatable dose dial member when in its locked state and permits rotation of the rotatable dose dial member when in its released state.

15. The drug delivery device according to claim 13, wherein the locking mechanism is biased into its locked state and is switched into its released state by placing the drug delivery device in the carry case.

16. The carry case according to claim 1, comprising a positioning sensor and/or encoder connected to the processor such that the electrically driven motor is operable to operate the electrically driven motor in a closed-loop positioning mode.

17. The drug delivery device according to claim 9, wherein the carry case is for storing the drug delivery device and the carry case comprises:
an outer shell;
a support recess for receiving and retaining the drug delivery device;
the driven coupling member;
an electrically driven motor;
a battery; and
processor connected with the electrically driven motor and the battery;
wherein the driven coupling member is configured to be driven by said electrically driven motor to rotate the rotatable dose dial member when engaged with the driven coupling member.

18. The drug delivery device according to claim 15, wherein the locking mechanism is switched into its released state by mechanical engagement of the rotatable dose dial member with the driven coupling member.
